(19) United States
(12) Patent Application Publication
(10) Pub. No.: US 2008/0131391 A1
(43) Pub. Date: Jun. 5, 2008

(54) COMPOSITIONS CONTAINING A QUATERNARY AMMONIUM POLYMER, A FATTY QUATERNARY AGENT AND A NONIONIC SURFACTANT

(75) Inventors: Angela Ellington, Flossmoor, IL (US); Carmen Patton, South Holland, IL (US); Nikisha Hunter, Chicago, IL (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/939,012

(22) Filed: Nov. 13, 2007

Related U.S. Application Data
(60) Provisional application No. 60/872,396, filed on Dec. 1, 2006.

(51) Int. Cl. A61K 8/72; A61Q 5/00

(52) U.S. Cl. 424/70.11

(57) ABSTRACT

A composition and process for treating a keratinous substrate involving contacting the keratinous substrate with a composition containing: a) at least one quaternary ammonium polymer; b) at least one fatty quaternary agent; c) at least one nonionic surfactant; and d) optionally, at least one ceramide, and wherein the composition is substantially anhydrous.

### STATEMENT OF RELATED APPLICATIONS

This application is based on and claims the benefit of U.S. Provisional Application Ser. No. 60/872,396, filed Dec. 1, 2006, the entire disclosure of which is incorporated by reference herein.

### BACKGROUND OF THE INVENTION

The present invention relates to an anhydrous composition based on a combination of at least one quaternary ammonium polymer, at least one fatty quaternary agent, and at least one nonionic surfactant. The present invention also relates to a process for treating hair based on an anhydrous composition that provides conditioning, moisturization, shine, and cosmetic feel, and helps reduce breakage.

Anhydrous hair care compositions such as pomades, for example, are usually made up of mineral oil and petrolatum and are used to enhance the feel and appearance of hair. With repeated use, however, these materials tend to leave an oily buildup and weigh hair down. Moreover, due to their high oil content, products of this nature tend to be difficult to rinse out of the hair.

A need therefore exists for a hair treatment composition and process that can reduce some of these undesirable effects and at the same time, can offer exceptional cosmetic feel and anti-frizz properties, plus restore moisturization to, and reduce breakage of, the hair.

### BRIEF SUMMARY OF THE INVENTION

The present invention relates to a cosmetic composition containing:

(a) at least one quaternary ammonium polymer;

(b) at least one fatty quaternary agent;

(c) at least one nonionic surfactant; and

(d) optionally, at least one ceramide; and

wherein the composition is substantially anhydrous.

The present invention also relates to a process for treating a keratinous substrate involving contacting the substrate with the above-disclosed cosmetic composition.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

As used herein, the expression “at least one” means one or more and thus includes individual components as well as mixtures/combinations.

The term “substantially anhydrous” means that the composition is either completely free of water or contains no appreciable amount of water, preferably no more than 5% by weight, and more preferably no more than 1% by weight, based on the weight of the composition.

“Cosmetically acceptable” means that the item in question is compatible with any keratin material. For example, “cosmetically acceptable medium” means a medium that is compatible with any keratinous substrate.

“Conditioning” as used herein means imparting to at least one keratinous fiber at least one property chosen from combability, manageability, moisture-retentivity, luster, shine, and softness. The state of conditioning is evaluated by measuring, and comparing, the ease of combability of the treated hair and of the untreated hair in terms of combing work (gm−in).

“Formed from,” as used herein, means obtained from chemical reaction of, wherein “chemical reaction,” includes spontaneous chemical reactions and induced chemical reactions. As used herein, the phrase “formed from”, is open ended and does not limit the components of the composition to those listed, e.g., as component (i) and component (ii). Furthermore, the phrase “formed from” does not limit the order of adding components to the composition or require that the listed components (e.g., components (i) and (ii)) be added to the composition before any other components.

“Silicone compound,” as used herein, includes, for example, silica, silanes, silazanes, siloxanes, and organosiloxanes; and refers to a compound comprising at least one silicon; wherein the silicone compound may be chosen from linear silicone compounds, branched silicone compounds, and cyclic silicone compounds; further wherein the silicone compound may optionally be substituted; and further wherein the silicone compound may optionally further comprise at least one heteroatom intercalated in the silicone chain, wherein the at least one heteroatom is different from the at least one silicon.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups.

“Keratinous substrate” as defined herein may be human keratinous fiber, and may be chosen from, for example, hair, eyelashes, and eyebrows, as well as the stratum corneum of the skin and nails.

“Polymers,” as defined herein, include homopolymers and copolymers formed from at least two different types of monomers.

The cosmetic compositions and methods of the present invention can comprise, consist of, or consist essentially of, the essential elements and limitations of the invention described herein, as well as any additional or optional ingredients, components, or limitations described herein or otherwise useful in personal care compositions intended for topical application to hair and skin.

The present invention relates to a cosmetic composition, preferably non-aqueous, for use in treating a keratinous substrate such as hair. The cosmetic composition is based on a mixture of at least one quaternary ammonium polymer, at least one fatty quaternary agent, at least one nonionic surfactant, and optionally, at least one ceramide. The present invention also relates to a process for treating hair based on an anhydrous composition involving contacting the substrate with the above-disclosed cosmetic composition.

Including water-soluble quaternary ammonium polymers and fatty quaternary agents in anhydrous compositions can be problematic in terms of formulating a stable composition. Given that many hair treatment and hair care compositions are emulsion aqueous based types of compositions, the incorporation of such ingredients into an anhydrous composition can be difficult.

It has been surprisingly found that the combination of a quaternary ammonium polymer, a fatty quaternary agent. and a nonionic surfactant resulted in a stable and substantially anhydrous composition that is homogeneous and clear to substantially clear.

Accordingly, one aspect of the present invention is a care and/or treatment composition for keratinous substrates such as hair which is able to address or overcome some or all of the aforementioned problems.

The present invention also relates to methods of conditioning, reducing breakage of and restoring moisture to, hair comprising applying a composition comprised of at least one quaternary ammonium polymer, at least one fatty quaternary agent, at least one nonionic surfactant, and optionally, at least one ceramide, to the hair in an amount sufficient to condition, reduce breakage of and restore moisture to hair.

The present invention also relates to methods of treating, caring for and/or enhancing the appearance of keratinous substrates comprising applying compositions of the present invention to the keratinous substrates in an amount sufficient to treat, care for and/or enhance the appearance of the keratinous substrates.

It is to be understood that both the foregoing general description and the following detailed description are exemplary and explanatory only, and are not restrictive of the invention.

The composition of the present invention may be in any form suitable for use on keratinous substrates and can be, for example, leave-in conditioners, hair styling compositions, hair treatment compositions, scalp treatments, etc.

As defined herein, the inventive composition is considered stable when all ingredients are miscible and the composition has resisted any aesthetic change (such as two-phase separation, syneresis, etc.) over a specified period.

Quaternary Ammonium Polymer

According to the present invention, compositions comprising at least one quaternary ammonium polymer are provided. The at least one quaternary ammonium polymer is a polymer comprising alkyl-substitued diamine monomers. For example, the quaternary ammonium polymer is preferably a polymer comprising repeating units of formula (a):

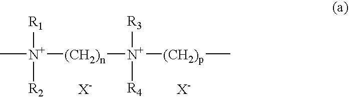

wherein:

R₁, R₂, R₃ and R₄, which may be identical or different, are each chosen from alkyl groups comprising from 1 to 4 carbon atoms and hydroxyalkyl groups comprising from 1 to 4 carbon atoms;

n and p, which may be identical or different, are each chosen from integers ranging from 2 to 20; and

X⁻ is an anion chosen from anions derived from inorganic acids and anions derived from organic acids.

Representative polymers of formula (a) include those in which R₁, R₂, R₃ and R₄ are chosen from methyl and ethyl groups and X⁻ is a halogen atom such as a halogen chosen from chlorine, iodine and bromine.

Further, representative polymers of formula (a) include polymers in which R₁, R₂, R₃ and R₄ are methyl groups and n=3, p=6 and X═Cl, such as those of which the molecular weight, determined by gel-permeation chromatography, ranges from 9500 to 9900 and exemplified by formula (W):

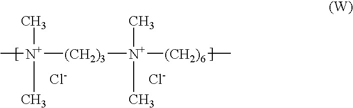

Other polymers of formula (a) include those where R₁ and R₂ are methyl groups, R₃ and R₄ are ethyl groups and n=p=3 and X═Br, such as those of which the molecular weight, determined by gel-permeation chromatography, is approximately 1200 and exemplified by formula (U):

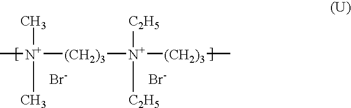

Such quaternary ammonium polymers of formula (a) can be prepared as described in French Patent 2,270,846, the entire disclosure of which is incorporated by reference herein.

Particularly preferred examples of the at least one quaternary ammonium polymer are polyquaternium-34 and Ionene G, also known as hexadimethrine chloride, which is a polymer of N,N,N′,N′-tetramethylhexamethylenediamine and trimethylene chloride and is disclosed in U.S. patent application publication no. 2002/0013972, the entire contents of which is hereby incorporated by reference.

Preferably, the at least one quaternary ammonium polymer is present in an amount of at least 0.001%, more preferably at least 0.1%, more preferably at least 0.2%, and more preferably at least 0.5% by weight with respect to the total weight of the composition. Preferred ranges are from 0.001% to 1.0% by weight of the total weight of the composition, more preferably from 0.1% to 1.0% of the total weight of the composition, more preferably from 0.1% to 0.5% of the total weight of the composition, and most preferably from 0.2% to 0.5%, including all ranges and subranges therebetween.

Fatty Quaternary Agents

The present invention provides for the use of fatty quaternary agents containing from about 6 to about 22 carbon atoms. The anion of the quaternary agent can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, benzyl or short chain alkyl or hydroxyalkyl groups such as methyl, ethyl, hydroxymethyl or hydroxyethyl, hydroxypropyl or combinations thereof.

Examples of fatty quaternary agents include, but are not limited to: Behentrimonium chloride, Cocotrimonium chloride, Cethethyldimonium bromide, Dibehenyldimonium chloride, Dihydrogenated tallow benzylmonium chloride, disoyadimonium chloride, Ditallowedimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowedimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, behentrimonium methosulfate (18-MEA), stearalkonium chloride, and mixtures thereof. Other quaternary ammonium compounds are listed in the CTFA Cosmetic Ingredient Handbook, First Edition, on pages 41-42, incorporated herein by reference.

A preferred fatty quaternary agent is Incroquat® Behenyl 18-MEA, which is a mixture of Behentrimonium Methosulfate, C10-40 Isoalkylamidopropylethyldimonium Ethosulfate (Quaternium-33) and Cetyl Alcohol, commercially available from Croda, Inc.

In the present invention, the at least one fatty quaternary agent is used in an amount of from 0.001% to 2.0% by weight, preferably from 0.01% to 1.0% by weight, and more preferably from 0.1% to 0.5% by weight, based on the total weight of the composition.

Nonionic Surfactant

According to the present invention, compositions comprising at least one nonionic surfactant are provided.

In general, nonionic surfactants having a Hydrophilic-Lipophilic Balance (HLB) of 4 or more, preferably from 4 to 20, are contemplated for use by the present invention. Non-limiting examples of nonionic surfactants useful in the compositions of the present invention are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C₁₂-C₅₀ range, preferably in the C16-C40 range, more preferably in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Paterson, N.J. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, e.g. C₈₋₃₀ alcohols, with sugar or starch polymers. These compounds can be represented by the formula (S)n--O—R wherein S is a sugar moiety such as glucose, fructose, mannose, galactose, and the like; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈₋₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from ICI Specialty Chemicals, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to about 20).

The preferred ethoxylated fatty alcohols include oleth-3 (an oleyl ethoxylate having an average degree of ethoxylation of 3), oleth-5 (an oleyl ethoxylate having an average degree of ethoxylation of 5), and oleth-20 (an oleyl ethoxylate having an average degree of ethoxylation of 20), all commercially available from Croda, Inc.

Preferred nonionic surfactants are those with an HLB of at least 4. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 4.

The nonionic surfactant is present in an amount ranging from 0.5% to 20% by weight, preferably from 1.0% to 10% by weight, and more preferably from 1.0% to 5.0% by weight, based on the total weight of the composition.

Ceramide

Optionally, ceramides may be used in the inventive composition for moisturizing the fiber and maintaining cuticle integrity. Ceramides are available by extraction from natural sources, or as synthetic ceramides and pseudoceramides. Preferred ceramides are chosen from 2-oleamido-1,3-octanediol and Ceramide II, ex Quest. Mixtures of ceramides may also be suitable, such as Ceramides LS, ex Laboratories Serobiologiques.

Preferably, the ceramide is present in an amount ranging from 0.001% to 0.5% by weight of the total weight of the composition, more preferably from 0.01% to 0.5% of the total weight of the composition, and most preferably from 0.01% to 0.1%, including all ranges and subranges therebetween.

Auxiliary Ingredients

Conditioning Agents

Suitable conditioning agents may be chosen from ester oils such as mono-, di- and tri-esters of glycerol. Preferred glyceride fatty esters are derived from carboxylic acids of carbon chain length ranging from C₆ to C₄₀, preferably from C₁₀ to C₂₂, and more preferably from C₁₂ to C₁₈.

Synthetic ester oils which may be used include, but are not limited to, trimyristin, triolein, tristearin and glyceryl dilaurate. Vegetable derived glyceride fatty esters are particularly preferred, and specific examples of preferred materials as sources of glyceride fatty esters include peanut oil, sesame oil, avocado oil, coconut, cocoa butter, almond oil, safflower oil, corn oil, cotton seed oil, castor oil, hydrogenated castor oil, olive oil, jojoba oil, palm oil, soybean oil, wheat germ oil, linseed oil, and sunflower seed oil. Coconut oil, sunflower oil, castor oil and mixtures thereof are particularly preferred.

Particularly useful glyceryl ester oils herein include caprylic/capric triglyceride with the tradename Miglyol812, from Degussa-Huls A G (Frankfurt, Germany), triisostearin with tradename SUN ESPOL G-318 available from Taiyo Kagaku, triolein with tradename CITHROL GTO available from Croda, Inc. (Parsippany, N.J., USA), trilinolein with tradename EFADERMA-F available from Vevy (Genova, Italy), or tradename EFA-GLYCERIDES from Brooks (South Plainfield, N.J., USA).

Additional examples of suitable esters include, for example, C12-15 alkyl benzoate (such as FINSOLV TN from Finetex, Elmwood Park, N.J.); octyl methoxy cinnamate (such as ESCALOL 557 from ISP, Wayne, N.J. (but in amounts less than 6% because of irritancy); isostearyl isostearate (such as (PRISORINE IS 2039 from Unichema, Chicago, Ill.); benzyl benzoate; 2,6-di-(ethylhexyl)naphthalate (such as Hallbrite TQ from the C.P. Hall Company, Bedford Park, Ill.); butyl octyl salicylate; glyceryl monostearate; n-dibutyl sebacate; isopropyl myristate; isopropyl palmitate; butyl stearate; cetyl lactate; isocetyl stearate; hexyl laurate; decyl oleate; isostearyl isostearate; ethyl hexyl maleate; sorbitan monoaurate; sorbitan monooleate; sorbitan sesquioleate; sorbitan trioleate; isopropyl palmitate; isopropyl stearate; stearyl stearate; diisopropyl adipate; diisopropyl sebacate; butyl myristate; isopropyl laurate; isotridecyl isononanoate; isostearyl neopentanoate; tridecyl neopentanoate; cetyl octanoate; cetyl ricinoleate; decyl isostearate; isodecyl oleate; isodecyl neopentanoate; isohexyl neopentanoate; tridecyl octanoate; alkyl benzoate; propyl myristate; propyl palmitate; tridecyl stearate; octyl palmitate and so on. Other suitable esters may also include esters such as tridecyl trimellilate, neopentyl glycol dicaprilate/dicaprate.

Also suitable may be synthetic or semi-synthetic glyceryl esters, e.g. fatty acid mono-, di-, and triglycerides which are natural fats or oils that have been modified, for example, acetylated castor oil, glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, PEG glyceryl tallowates, and so on.

The at least one conditioning agent may be employed in the present composition in an amount of from greater than 0 to 95% by weight; greater than 0 to 50% by weight; greater than 0 to 25% by weight; greater than 0 to 15% by weight; greater than 0 to 10% by weight; greater than 0 to 5% by weight, based on the total weight of the composition.

Other conditioning agents may be chosen from natural, synthetic, saturated and unsaturated oils. Mention may be made of mineral oil, linear, branched and/or cyclic alkanes which may be volatile and, in particular, liquid paraffin, liquid petroleum jelly or hydrogenated polyisobutylene, isododecane or “Isopars”, volatile isoparaffins.

Mention may also be made of aliphatic fatty liquid monoalcohols containing 6 to 40 carbon atoms, the hydrocarbon-based chain not comprising a substitution group. Monoalcohols according to the invention that may be mentioned include oleyl alcohol, decanol, octyldodecanol and linoleyl alcohol.

Mention may also be made of silicone oils such as polydimethylsiloxanes and polymethylphenylsiloxanes, optionally substituted with aliphatic and/or aromatic groups, which are optionally fluorinated, or with functional groups such as hydroxyl, thiol and/or amine groups, and volatile silicone oils, which are especially cyclic.

In particular, mention may be made of volatile and/or non-volatile, optionally branched silicone oils. The term “volatile oil” means an oil capable of evaporating from the skin or the lips in less than one hour, and especially having a vapor pressure, at room temperature and atmospheric pressure, ranging from 10⁻³ to 300 mmHg (0.13 Pa to 40,000 Pa).

As volatile silicone oils that may be used in the invention, mention may be made of linear or cyclic silicones containing from 2 to 7 silicon atoms, these silicones optionally comprising alkyl or alkoxy groups containing from 1 to 10 carbon atoms. Mention may be made in particular of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Among the non-volatile silicone oils that may be mentioned are non-volatile polydialkylsiloxanes, such as non-volatile polydimethylsiloxanes (PDMS); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyl trimethyl-siloxy diphenylsiloxanes, diphenyl dimethicones, diphenyl methyldiphenyltrisiloxanes and polymethylphenylsiloxanes; polysiloxanes modified with fatty acids (especially of C₈-C₂₀), fatty alcohols (especially of C₈-C₂₀) or polyoxyalkylenes (especially polyoxy-ethylene and/or polyoxypropylene); amino polysiloxanes; polysiloxanes containing hydroxyl groups; fluoro poly-siloxanes comprising a fluorinated group that is pendent or at the end of a silicone chain, containing from 1 to 12 carbon atoms, all or some of the hydrogen atoms of which are replaced with fluorine atoms; and mixtures thereof.

Additional ingredients in the composition may include, but are not limited to alcohol esters, vitamin derivatives, colorants, and perfumes.

Adjuvants

The compositions of the present invention may also contain adjuvants suitable for hair care. Suitable hair care adjuvants include, but are not limited to:

Natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred combination of natural hair root nutrients for inclusion in compositions of the invention is isoleucine and glucose. A particularly preferred amino acid nutrient is arginine.

Nonvolatile silicone compounds may also be employed as adjuvants in the present invention such as, for example, a polydimethylsiloxane, polyalkyl siloxane, a polyaryl siloxane or a polyalkylaryl siloxane. The nonvolatile silicones are nonfunctional siloxanes or siloxane mixtures having a viscosity of about 10 to about 10,000 cst, and most preferred viscosity about 10 to 500 cst at 25° C. A nonvolatile silicone compound is described as having a boiling point at atmospheric pressure of greater than about 250° C. A particularly preferred nonvolatile silicone compound is cyclopentasiloxane, commercially available from Dow Corning under the tradename DC 245.

Additional adjuvants include gelling agents, waxes, preservatives, suspending agents, volatile solvents, thickening agents, film formers, spreading agent, dispersants, antifoaming agents, wetting agents, UV-screening agents, antioxidants, perfumes, fillers, active agents, moisturizers, vitamins and their derivatives, biological materials, and derivatives of any of the foregoing.

The composition of the present invention possesses good conditioning and anti-frizz properties, reduces breakage of hair and provides shine to hair.

According to another embodiment of the present invention, there is also provided a process for treating a keratinous substrate, such as hair, involving applying the above-disclosed composition onto the substrate. The process facilitates the styling and conditioning of substrates, such as hair, in a cosmetically appealing manner.

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

### EXAMPLES

### Example 1

| MINERAL OIL | qs |
|---|---|
| MINERAL OIL (and) HYDROGENATED | 47.00 |
| ETHYLENE/PROPYLENE/STYRENE | |
| COPOLYMER (and) HYDROGENATED | |
| BUTYLENE/ETHYLENE/STYRENE COPOLYMER | |
| C12-15 ALKYL BENZOATE | 2.00 |
| OLETH-5 | 1.00 |
| CETYL ALCOHOL (and) | 0.01 |
| BEHENTRIMONIUM METHOSULFATE (and) | |
| QUATERNIUM-33* | |
| 2-OLEAMIDO-1,3-OCTADECANEDIOL | 0.001 |
| HEXADIMETHRINE CHLORIDE | 0.001 |

*also known as C10-40 Isoalkylamidopropylethyldimonium Ethosulfate

### Example 2

| MINERAL OIL | qs |
|---|---|
| OLETH-5 | 5.00 |
| CETYL ALCOHOL (and) BEHENTRIMONIUM | 1.00 |
| METHOSULFATE (and) | |
| QUATERNIUM-33 | |
| 2-OLEAMIDO-1,3-OCTADECANEDIOL | 0.10 |
| OLETH-20 | 5.00 |
| HEXADIMETHRINE CHLORIDE | 0.50 |

### Example 3

| MINERAL OIL | qs |
|---|---|
| OLETH-3 | 5.00 |
| CETYL ALCOHOL (and) BEHENTRIMONIUM | 1.00 |
| METHOSULFATE (and) | |
| QUATERNIUM-33 | |
| 2-OLEAMIDO-1,3-OCTADECANEDIOL | 0.10 |
| OLETH-20 | 5.00 |
| HEXADIMETHRINE CHLORIDE | 0.50 |

### Example 4

| MINERAL OIL | qs |
|---|---|
| OLETH-5 | 5.00 |
| CETYL ALCOHOL (and) BEHENTRIMONIUM | 0.50 |
| METHOSULFATE (and) | |
| QUATERNIUM-33 | |
| 2-OLEAMIDO-1,3-OCTADECANEDIOL | 0.10 |
| OLETH-20 | 5.00 |
| HEXADIMETHRINE CHLORIDE | 0.20 |

What is claimed is:

1. A process for treating a keratinous substrate comprising contacting the keratinous substrate with a composition containing:
a) at least one quaternary ammonium polymer;
b) at least one fatty quaternary agent;
c) at least one nonionic surfactant; and
d) optionally, at least one ceramide; and
wherein the composition is substantially anhydrous.

2. The process of claim 1 wherein the at least one quaternary ammonium polymer is chosen from polyquaternium-34, hexadimethrine chloride, and mixtures thereof.

3. The process of claim 1 wherein the at least one quaternary ammonium polymer is present in an amount of from about 0.001% to about 1.0% by weight of the total weight of the composition.

4. The process of claim 1 wherein the at least one quaternary ammonium polymer is present in an amount of from about 0.2% to about 0.5% by weight of the total weight of the composition.

5. The process of claim 1 wherein the at least one fatty quaternary agent contains from about 6 to about 22 carbon atoms.

6. The process of claim 1 wherein the at least one fatty quaternary agent is a mixture of Behentrimonium Methosulfate, C10-40 Isoalkylamidopropylethyldimonium Ethosulfate and Cetyl Alcohol.

7. The process of claim 1 wherein the at least one fatty quaternary agent is present in an amount of from about 0.001% to about 2.0% by weight of the total weight of the composition.

8. The process of claim 1 wherein the at least one fatty quaternary agent is present in an amount of from about 0.1% to about 0.5% by weight of the total weight of the composition.

9. The process of claim 1 wherein the at least one nonionic surfactant is chosen from oleth-3, oleth-5 and oleth-20, and mixtures thereof.

10. The process of claim 1 wherein the at least one nonionic surfactant is present in an amount of from about 0.5% to about 20% by weight of the total weight of the composition.

11. The process of claim 1 wherein the at least one nonionic surfactant is present in an amount of from about 1.0% to about 5.0% by weight of the total weight of the composition.

12. The process of claim 1 wherein the at least one ceramide is present in an amount of from about 0.001% to about 0.5% by weight of the total weight of the composition.

13. The process of claim 1 wherein the keratinous substrate is hair.

14. A cosmetic composition comprising:
a) at least one quaternary ammonium polymer;
b) at least one fatty quaternary agent;
c) at least one nonionic surfactant; and
d) optionally, at least one ceramide; and
wherein the composition is substantially anhydrous.

15. The composition of claim 14 wherein the at least one quaternary ammonium polymer is chosen from polyquaternium-34, hexadimethrine chloride, and mixtures thereof.

16. The composition of claim 14 wherein the at least one quaternary ammonium polymer is present in an amount of from about 0.001% to about 1.0% by weight of the total weight of the composition.

17. The composition of claim 14 wherein the at least one quaternary ammonium polymer is present in an amount of from about 0.2% to about 0.5% by weight of the total weight of the composition.

18. The composition of claim 14 wherein the at least one fatty quaternary agent is a mixture of Behentrimonium Methosulfate, C10-40 Isoalkylamidopropylethyldimonium Ethosulfate and Cetyl Alcohol.

19. The composition of claim 14 wherein the at least one fatty quaternary agent is present in an amount of from about 0.001% to about 2.0% by weight of the total weight of the composition.

20. The composition of claim 14 wherein the at least one fatty quaternary agent is present in an amount of from about 0.1% to about 0.5% by weight of the total weight of the composition.

21. The composition of claim 14 wherein the at least one nonionic surfactant is chosen from oleth-3, oleth-5 and oleth-20, and mixtures thereof.

22. The composition of claim 14 wherein the at least one nonionic surfactant is present in an amount of from about 0.5% to about 20% by weight of the total weight of the composition.

23. The composition of claim 14 wherein the at least one nonionic surfactant is present in an amount of from about 1.0% to about 5.0% by weight of the total weight of the composition.

24. The composition of claim 14 wherein the at least one ceramide is present in an amount of from about 0.001% to about 0.5% by weight of the total weight of the composition.
